(19) United States
(12) Patent Application Publication
(10) Pub. No.: US 2026/0274973 A1
(43) Pub. Date: Sep. 17, 2026

(54) METHODS FOR REDUCING EOSINOPHILIC INFILTRATION BY ADMINISTERING AN ANTI-INTERLEUKIN-4 RECEPTOR (IL4R) ANTIBODY

(71) Applicant: Regeneron Pharmaceuticals, Inc., Tarrytown, NY (US)

(72) Inventors: Ana Kostic, New York, NY (US); Ludmila Kelly, Silver Spring, MD (US); Xia Liu, Pleasantville, NY (US); Brendan J. Classon, Seattle, WA (US); Allen Radin, New York, NY (US)

(21) Appl. No.: 19/686,394

(22) Filed: May 22, 2026

Related U.S. Application Data
(63) Continuation of application No. 19/169,892, filed on Apr. 3, 2025.
Application No. 19/169,892 is a continuation of application No. 17/864,000, filed on Jul. 13, 2022, now Pat. No. 12,291,571.
Application No. 17/864,000 is a continuation of application No. 16/910,884, filed on Jun. 24, 2020, now Pat. No. 11,421,036.
Application No. 16/910,884 is a continuation of application No. 15/017,664, filed on Feb. 7, 2016, now Pat. No. 10,730,948.
Application No. 15/017,664 is a continuation of application No. 14/328,336, filed on Jul. 10, 2014, now Pat. No. 9,290,574.
(60) Provisional application No. 61/844,978, filed on Jul. 11, 2013.

(51) Int. Cl. C07K 16/28; A61K 9/00; A61K 31/56; A61K 39/00; A61K 39/395; A61K 45/06

(52) U.S. Cl. CPC C07K 16/2866; A61K 9/0019; A61K 31/56; A61K 39/3955; A61K 45/06; A61K 2039/505; A61K 2039/54; A61K 2039/545; A61K 2039/577; C07K 2317/21; C07K 2317/565; C07K 2317/76; C07K 2318/00

(57) ABSTRACT

The present invention provides methods for treating, preventing or reducing the severity of eosinophilic esophagitis. The methods of the present invention comprise administering to a subject in need thereof a therapeutic composition comprising an interleukin-4 receptor (IL-4Rα) inhibitor such as an anti-IL-4Rα antibody.

### CROSS-REFERENCE TO RELATED APPLICATIONS

This application is a continuation of U.S. patent application Ser. No. 19/169,892, filed on Apr. 3, 2025, which is a continuation of U.S. patent application Ser. No. 17/864,000, filed on Jul. 13, 2022, which is a continuation of U.S. patent application Ser. No. 16/910,884, filed on Jun. 24, 2020, which is a continuation of U.S. patent application Ser. No. 15/017,664, filed on Feb. 7, 2016, now U.S. Pat. No. 10,730,948, issued on Aug. 4, 2020, which is a continuation of U.S. patent application Ser. No. 14/328,336, filed on Jul. 10, 2014, now U.S. Pat. No. 9,290,574, issued on Mar. 22, 2016, which claims the benefit under 35 U.S.C. § 119 (e) of U.S. Provisional Application No. 61/844,978, filed on Jul. 11, 2013, the disclosures of which are herein incorporated by reference in their entirety.

### SEQUENCE LISTING

This application includes a Sequence Listing in electronic format entitled “40848-0074USC3_SL.xml” which was created Dec. 19, 2022, and which has a size of 19,338 bytes. The contents of the file “40848-0074USC3_SL.xml” are incorporated by reference herein.

### FIELD OF THE INVENTION

The present invention relates to the use of interleukin-4 receptor inhibitors to treat or prevent eosinophilic esophagitis in a subject in need thereof.

### BACKGROUND

Eosinophilic esophagitis (EoE) is an emerging disease characterized by esophageal dysfunction and by abnormal eosinophilic inflammation of the esophagus. The typical symptoms of EoE include food refusal, vomiting, heartburn, dysphagia and food impaction, which may lead to impaired quality of life. EoE is found to be associated with food allergy in many patients. Some patients may also have concomitant asthma or an atopic disease such as atopic dermatitis, or allergic rhinitis. EoE is currently diagnosed by endoscopy of the esophagus and biopsy of the esophageal tissue to check for eosinophilia. Treatment options are currently limited to allergen withdrawal, diet modification and corticosteroids. Accordingly, an unmet need exists in the art for effective therapeutic approaches without adverse side-effects that prevent or treat eosinophilic esophagitis and prevent relapse.

### BRIEF SUMMARY OF THE INVENTION

According to one aspect of the present invention, methods are provided for treating, preventing or ameliorating at least one symptom or indication of eosinophilic esophagitis (EoE) in a subject. The methods according to this aspect of the invention comprise administering a therapeutically effective amount of a pharmaceutical composition comprising an interleukin-4 receptor (IL-4R) inhibitor to a subject in need thereof. In certain embodiments, the subject in need thereof exhibits an allergic reaction to a food allergen or a non-food allergen.

According to another aspect of the present invention, methods are provided for reducing the level of an EoE-associated biomarker in a subject. In certain embodiments, the EoE-associated biomarker is selected from the group consisting of, e.g., circulating or esophagus eosinophils, eotaxin-3, periostin, serum IgE (total and allergen-specific), IL-13, IL-5, serum thymus and activation regulated chemokine (TARC; CCL17), thymic stromal lymphopoietin (TSLP), serum eosinophilic cationic protein (ECP), and eosinophil-derived neurotoxin (EDN). The methods comprise administering a therapeutically effective amount of a pharmaceutical composition comprising an IL-4R inhibitor.

According to another aspect of the present invention, methods are provided for reducing the eosinophilic infiltration of esophagus in a subject in need thereof. In certain embodiments, methods are provided for reducing inflammation in the esophagus. The methods comprise administering a therapeutically effective amount of a pharmaceutical composition comprising an IL-4R inhibitor. In certain embodiments, the eosinophilic infiltration of the esophagus is represented by greater than or equal to about 15 eosinophils per high powered field in the esophagus of the subject in need thereof. In certain embodiments, the number of eosinophils is reduced by about 50% by day 10 following administration of the IL-4R inhibitor.

In certain embodiments, the IL-4R inhibitor is administered in combination with a second therapeutic agent or therapy.

In certain embodiments, the subject in need thereof has a concurrent disease or disorder selected from the group consisting of food allergy, atopic dermatitis, asthma, allergic rhinitis, allergic conjunctivitis and inherited connective tissue disorders.

Exemplary IL-4R inhibitors that can be used in the context of the methods of the present invention include, e.g., small molecule chemical inhibitors of IL-4R or its ligands (IL-4 and/or IL-13), or biological agents that target IL-4R or its ligands. According to certain embodiments, the IL-4R inhibitor is an antibody or antigen-binding protein that binds the IL-4Rα chain and blocks signaling by IL-4, IL-13, or both IL-4 and IL-13. In certain embodiments, the anti-IL-4R antibody or antigen-binding protein comprises the heavy chain complementarity determining regions (HCDRs) of a heavy chain variable region (HCVR) comprising the amino acid sequence of SEQ ID NO: 1 and the light chain CDRs of a light chain variable region (LCVR) comprising the amino acid sequence of SEQ ID NO: 2. One such type of antigen-binding protein that can be used in the context of the methods of the present invention is an anti-IL-4Rα antibody such as dupilumab.

In certain embodiments, the present invention provides use of an antibody or antigen-binding fragment thereof of the invention in the manufacture of a medicament to treat or inhibit or prevent eosinophilic esophagitis in a subject, including humans.

Other embodiments of the present invention will become apparent from a review of the ensuing detailed description.

### BRIEF DESCRIPTION OF THE FIGURES

FIG. 1 shows the serum IgE levels in mice injected with II25 DNA using the hydrodynamic DNA delivery (HDD) method and subsequently treated with the isotype control, anti-mIL-4R mAb or IL-13Ra2-mFc as described in Example 1 herein.

FIG. 2 shows the esophageal histology scores of mice injected with II25 DNA using the HDD method and subsequently treated with the isotype control, anti-mIL-4R mAb or IL-13Ra2-mFc as described in Example 1 herein.

FIG. 3 shows the esophageal histology scores (as described elsewhere herein) of mice sensitized by phosphate-buffered saline (PBS) or by peanut allergen extract (PAE) and challenged by PBS or PAE. The mice were treated with anti-mIL-4R mAb or an isotype control.

FIGS. 4A and 4B show serum levels of (4A) peanut allergen specific IgG1 and (4B) IgE in mice sensitized by PBS or by peanut allergen extract (PAE) and challenged by PBS or PAE. The mice were treated with anti-mIL-4R mAb or an isotype control.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Methods for Treating, Preventing or Ameliorating Eosinophilic Esophagitis

The present invention includes methods for treating, preventing, or ameliorating at least one symptom or indication of eosinophilic esophagitis (EoE) in a subject. The methods according to this aspect of the invention comprise administering a therapeutically effective amount of a pharmaceutical composition comprising an IL-4R inhibitor to the subject in need thereof. As used herein, the terms “treat”, “treating”, or the like, mean to alleviate symptoms, eliminate the causation of symptoms either on a temporary or permanent basis, or to prevent or slow the appearance of symptoms of eosinophilic inflammation in the esophagus. In certain embodiments, the present methods are useful for treating or ameliorating at least one symptom or indication of EoE including, but not limited to, eosinophilic infiltration of the esophagus, thickening of the esophageal wall, inflammation in the esophagus, appearance of trachea-like rings or ridges in the esophagus, chest and abdominal pain, food refusal, vomiting, dysphagia and food impaction.

“Eosinophilic Esophagitis” (EoE), as used herein, means an inflammatory disease characterized by abnormal eosinophilic inflammation within the esophagus and esophageal dysfunction. The primary symptoms of EoE include, but are not limited to, chest and abdominal pain, dysphagia, heartburn, food refusal, vomiting and food impaction. The clinicopathology of EoE is characterized by presence of ridges or trachea-like rings in the esophageal wall and eosinophilic infiltration in the esophageal mucosa. EoE is presently diagnosed by endoscopy of the esophagus followed by microscopic and biochemical analysis of the esophageal mucosal lining. EoE may be classified as allergic or non-allergic depending upon the status of the subject. The present invention includes methods to treat both allergic and non-allergic forms of EoE.

As used herein, the expression “a subject in need thereof” means a human or non-human mammal that exhibits one or more symptoms or indications of eosinophilic esophagitis, and/or who has been diagnosed with eosinophilic esophagitis (EoE). In certain embodiments, the methods of the present invention may be used to treat patients that show elevated levels of one or more EoE-associated biomarkers (described elsewhere herein). For example, the methods of the present invention comprise administering an IL-4R inhibitor to patients with elevated levels of IgE or eotaxin-3. The term “a subject in need thereof” may also include, e.g., subjects who, prior to treatment, exhibit (or have exhibited) one or more indications of EoE such as, e.g., esophageal overexpression of pro-inflammatory mediators such as mast cells, eosinophilic infiltration of the esophagus, thickening of the esophageal wall, dysphagia, food impaction and chest and abdominal pain and/or an elevated level of a EoE-associated biomarker. The term also includes subjects who show the presence of ≥15 eosinophils per high power field in the esophagus, and subjects with elevated peripheral eosinophil counts (>300 cells/μl) or elevated serum IgE (>150 kU/L).

In certain embodiments, the present methods may be used to treat subjects who exhibit pathology and symptoms that are observed in subjects with chronic esophagitis including in gastroesophageal reflux disease (GERD). In certain embodiments, the term “a subject in need thereof” includes subjects that are non-responsive to or resistant to anti-GERD therapy. For example, the present methods may be used to treat subjects that are resistant to proton pump inhibitors (PPI).

In the context of the present invention, “a subject in need thereof” may include a subset of population that is more susceptible to EoE or may show an elevated level of an EoE-associated biomarker. For example, “a subject in need thereof” may include a subject suffering from an atopic disease or disorder such as food allergy, atopic dermatitis, asthma, allergic rhinitis and allergic conjunctivitis. In certain embodiments, the term “a subject in need thereof” includes a subject who, prior to or at the time of administration of the IL-4R inhibitor, has or is diagnosed with a disease or disorder selected from the group consisting of atopic dermatitis, asthma, allergic rhinitis and allergic conjunctivitis. In certain embodiments, the term “a subject in need thereof” may include patients with inherited connective tissue disorders. Such a subject population may show an elevated level of an EoE-associated biomarker such as, e.g., IgE, eotaxin-3, periostin, IL-5, or IL-13.

In certain embodiments, “a subject in need thereof” includes a subject susceptible to an allergen. For example, “a subject in need thereof” includes a subject who may exhibit one of the following characteristics: (a) is prone to allergic reactions or responses when exposed to one or more allergens; (b) has previously exhibited an allergic response or reaction to one or more allergens; (c) has a known history of allergies; and/or (d) exhibits a sign or symptom of an allergic response or anaphylaxis. In certain embodiments, the subject is allergic to an allergen associated with EoE or that renders the subject susceptible and/or prone to developing EoE.

The term “allergen,” as used herein, includes any substance, chemical, particle or composition that is capable of stimulating an allergic response in a susceptible individual. Allergens may be contained within or derived from a food item such as, e.g., dairy products (e.g., cow's milk), egg, wheat, soy, corn, rye, fish, shellfish, peanuts and tree nuts. Alternatively, an allergen may be contained within or derived from a non-food item such as, e.g., dust (e.g., containing dust mite), pollen, insect venom (e.g., venom of bees, wasps, mosquitoes, etc.), mold, animal dander, latex, medication, drugs, ragweed, grass and birch.

In certain embodiments, the term “a subject in need thereof” includes a subset of population that exhibits an allergic reaction to a food allergen. For example, “a subject in need thereof” may include a subject who has an allergy to an allergen contained in a food item including, but not limited to, a dairy product, egg, wheat, soy, corn, rye, fish, shellfish, peanut, a tree nut, beef, chicken, oat, barley, pork, green beans, and fruits such as apple and pineapple.

In certain embodiments, the term includes a subject allergic to a non-food allergen such as allergens derived from dust, mold, insects, plants including pollen, and pets such as cats and dogs. Examples of non-food allergens (also known as environmental allergens or aeroallergens) include, but are not limited to, house dust mite allergens, pollen allergens, animal dander allergens, insect venom, grass allergens, and latex.

As used herein, the phrases “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production, increased allergen-specific immunoglobulin production and/or eosinophilia.

In some embodiments, the methods herein may be used to treat EoE in children who are ≤3 years old. For example, the present methods may be used to treat infants who are less than 1 month, 2 months, 3 months, 4 months, 5 months, 6 months, 7 months, 8 months, 9 months, 10 months, 11 months or less than 12 months old. In other embodiments, the methods of the present invention may be used to treat children who are more than 3 years old, more than 4 years, 5 years, 6 years, 7 years, 8 years, 9 years, 10 years, 11 years, 12 years, 13 years, 14 years, or more than 15 years old.

The present invention also includes methods for reducing eosinophilic infiltration. The methods according to this aspect of the invention comprise administering to the subject one or more doses of a pharmaceutical composition comprising an IL-4R inhibitor to reduce or eliminate the number of eosinophils, e.g., in the esophageal mucosa.

As used herein, “eosinophilic infiltration” refers to the presence of eosinophils in an organ or tissue including blood, esophagus, stomach, duodenum, and ileum of a subject. In the context of the invention, the term “eosinophilic infiltration” refers to presence of eosinophils in the mucosal lining of a region of the gastro-intestinal tract including, but not limited to, esophagus and stomach. Eosinophilic infiltration is analyzed, for example, in an esophageal tissue biopsy of a subject suffering from EoE. According to particular embodiments, “eosinophilic infiltration” refers to the presence of ≥15 eosinophils per high power field in the esophagus. The term “high power field” refers to a standard total magnification of 400× by a microscope used to view eosinophils in a tissue, e.g., from the esophagus of a subject. In certain embodiments, “eosinophilic infiltration” includes infiltration into a tissue by leucocytes, for example, lymphocytes, neutrophils and mast cells. The leucocyte infiltration into, e.g., esophageal tissue can be detected by cell surface markers such as eosinophil-specific markers (e.g., CD11c^{Low/Neg}, SiglecF⁺, F4/80⁺, EMR1⁺, Siglec 8⁺, and MBP2⁺), macrophage-specific markers (e.g., CD11b⁺, F4/80⁺, CD14⁺, EMR1⁺, and CD68⁺), neutrophil-specific markers (e.g., CD11b⁺, Ly6G⁺, Ly6C⁺, CD11b⁺, and CD66b⁺), and T-cell-specific markers (e.g., CD3⁺ CD4⁺ CD8⁺).

As used herein, a reduction in esophagus eosinophils means that the number of eosinophils and other leucocytes measured in the esophagus of a subject with EoE and who has been treated with an IL-4R inhibitor, is at least 5%, 10%, 20%, 50%, 70%, 80%, or 90% lower than the esophagus eosinophils measured in the same or an equivalent subject that has not been treated with the IL-4R inhibitor. In certain embodiments, reducing eosinophilic infiltration means detecting less than 15 eosinophils per high power field, more preferably less than 10 eosinophils, less than 9 eosinophils, less than 8 eosinophils, less than 7 eosinophils, less than 6 eosinophils, or less than 5 eosinophils per high power field in a biopsy of the esophageal mucosa. In certain embodiments, a reduction in esophagus eosinophils means that no eosinophils are detected in the esophageal mucosa of a subject.

The present invention includes methods for treating, preventing or reducing the severity of eosinophilic esophagitis comprising administering a therapeutically effective amount of a pharmaceutical composition comprising an IL-4R inhibitor to a subject in need thereof, wherein the pharmaceutical composition is administered to the subject in multiple doses, e.g., as part of a specific therapeutic dosing regimen. For example, the therapeutic dosing regimen may comprise administering multiple doses of the pharmaceutical composition to the subject at a frequency of about once a day, once every two days, once every three days, once every four days, once every five days, once every six days, once a week, once every two weeks, once every three weeks, once every four weeks, once a month, once every two months, once every three months, once every four months, or less frequently.

The methods of the present invention, according to certain embodiments, comprise administering to a subject a therapeutically effective amount of a pharmaceutical composition comprising an IL-4R inhibitor in combination with a second therapeutic agent. The second therapeutic agent may be an agent selected from the group consisting of, e.g., an IL-1beta inhibitor, an IL-5 inhibitor, an IL-9 inhibitor, an IL-13 inhibitor, an IL-17 inhibitor, an IL-25 inhibitor, a TNFalpha inhibitor, an eotaxin-3 inhibitor, an IgE inhibitor, a prostaglandin D2 inhibitor, an immunosuppressant, a corticosteroid, a glucocorticoid, a proton pump inhibitor, a decongestant, an antihistamine, and a non-steroidal anti-inflammatory drug (NSAID). In certain embodiments, the IL-4R inhibitor of the invention may be administered in combination with therapy including allergen removal and diet management. As used herein, the phrase ‘in combination with” means that the pharmaceutical composition comprising an IL-4R inhibitor is administered to the subject at the same time as, just before, or just after administration of the second therapeutic agent. In certain embodiments, the second therapeutic agent is administered as a co-formulation with the IL-4R inhibitor. In a related embodiment, the present invention includes methods comprising administering a therapeutically effective amount of a pharmaceutical composition comprising an IL-4R inhibitor to a subject who is on a background anti-allergy therapeutic regimen. The background anti-allergy therapeutic regimen may comprise a course of administration of, e.g., steroids, antihistamines, decongestants, anti-IgE agents, etc. The IL-4R inhibitor may be added on top of the background anti-allergy therapeutic regimen. In some embodiments, the IL-4R inhibitor is added as part of a “background step-down” scheme, wherein the background anti-allergy therapy is gradually withdrawn from the subject over time (e.g., in a stepwise fashion) while the IL-4R inhibitor is administered the subject at a constant dose, or at an increasing dose, or at a decreasing dose, over time.

### Eosinophilic Esophagitis-Associated Biomarkers

The present invention also includes methods involving the use, quantification, and analysis of EoE-associated biomarkers. As used herein, the term “EoE-associated biomarker” means any biological response, cell type, parameter, protein, polypeptide, enzyme, enzyme activity, metabolite, nucleic acid, carbohydrate, or other biomolecule which is present or detectable in an EoE patient at a level or amount that is different from (e.g., greater than or less than) the level or amount of the marker present or detectable in a non-EoE patient. Exemplary EoE-associated biomarkers include, but are not limited to, e.g., esophagus eosinophils, eotaxin-3 (CCL26), periostin, serum IgE (total and allergen-specific), IL-13, IL-5, serum thymus and activation regulated chemokine (TARC; CCL17), thymic stromal lymphopoietin (TSLP), serum eosinophilic cationic protein (ECP), and eosinophil-derived neurotoxin (EDN). The term “EoE-associated biomarker” also includes a gene or gene probe known in the art, which is differentially expressed in a subject with EoE as compared to a subject without EoE. For example, genes which are significantly up-regulated in a subject with EoE include, but are not limited to, T-helper 2 (Th2)-associated chemokines such as CCL8, CCL23 and CCL26, periostin, cadherin-like-26, and TNFα-induced protein 6 (Blanchard et al 2006, J. Clin. Invest. 116:536-547). Alternatively, “EoE-associated biomarker” also includes genes that are down regulated due to EoE such as terminal differentiation proteins (e.g., filaggrin) (Blanchard et al 2006, J. Clin. Invest. 116:536-547). Certain embodiments of the invention relate to use of these biomarkers for monitoring disease reversal with the administration of the IL-4R antagonist. Methods for detecting and/or quantifying such EoE-associated biomarkers are known in the art; kits for measuring such EoE-associated biomarkers are available from various commercial sources; and various commercial diagnostic laboratories offer services that provide measurements of such biomarkers as well.

According to certain aspects of the invention, methods for treating EoE are provided which comprise: (a) selecting a subject who exhibits a level of at least one EoE-associated biomarker prior to or at the time of treatment, which signifies the disease state; and (b) administering to the subject a pharmaceutical composition comprising a therapeutically effective amount of an IL-4R antagonist. In certain embodiments of this aspect of the invention, the subject is selected on the basis of an elevated level of IgE or eotaxin-3.

According to other aspects of the invention, methods for treating EoE are provided which comprise administering to a subject a pharmaceutical composition comprising a therapeutically effective amount of an IL-4R antagonist, wherein administration of the pharmaceutical composition to the subject results in a decrease in at least one EoE-associated biomarker (e.g., esophagus eosinophils, eotaxin-3, IgE, etc.) at a time after administration of the pharmaceutical composition, as compared to the level of the biomarker in the subject prior to the administration.

As will be appreciated by a person of ordinary skill in the art, an increase or decrease in an EoE-associated biomarker can be determined by comparing (i) the level of the biomarker measured in a subject at a defined time point after administration of the pharmaceutical composition comprising an IL-4R antagonist to (ii) the level of the biomarker measured in the patient prior to the administration of the pharmaceutical composition comprising an IL-4R antagonist (i.e., the “baseline measurement”). The defined time point at which the biomarker is measured can be, e.g., at about 4 hours, 8 hours, 12 hours, 1 day, 2 days, 3 days, 4 days, 5 days, 6 days, 7 days, 8 days, 9 days, 10 days, 15 days, 20 days, 35 days, 40 days, 50 days, 55 days, 60 days, 65 days, 70 days, 75 days, 80 days, 85 days, or more after administration of the of the pharmaceutical composition comprising an IL-4R antagonist.

According to certain embodiments of the present invention, a subject may exhibit a decrease in the level of one or more of IgE and/or eotaxin-3 following administration of a pharmaceutical composition comprising an IL-4R antagonist (e.g., an anti-IL-4R antibody). For example, at about day 1, day 4, day 8, day 15, day 22, day 25, day 29, day 36, day 43, day 50, day 57, day 64, day 71 or day 85, following administration of a first, second, third or fourth dose of a pharmaceutical composition comprising about 75 mg to about 600 mg of an anti-IL-4R antibody (e.g., dupilumab), the subject, according to the present invention, may exhibit a decrease in eotaxin-3 of about 1%, 2%, 5%, 10%, 15%, 20%, 25%, 30%, 35%, 40%, 45%, 50%, 55%, 60%, 65%, 70%, 75%, 80%, 85%, 90%, 95% or more from baseline (wherein “baseline” is defined as the level of eotaxin-3 in the subject just prior to the first administration). Similarly, at about day 1, day 4, day 8, day 15, day 22, day 25, day 29, day 36, day 43, day 50, day 57, day 64, day 71 or day 85, following administration of a first, second, third or fourth dose of a pharmaceutical composition comprising about 75 mg to about 600 mg of an anti-IL-4R antibody (e.g., dupilumab), the subject, according to the present invention, may exhibit a decrease in IgE of about 1%, 2%, 5%, 10%, 15%, 20%, 25%, 30%, 35%, 40%, 45%, 50%, 55%, 60%, 65%, 70%, 75%, 80%, 85%, 90%, 95% or more from baseline (wherein “baseline” is defined as the level of IgE in the subject just prior to the first administration).

The present invention also includes methods for determining whether a subject is a suitable subject for whom administration of a pharmaceutical composition comprising an IL-4R antagonist would be beneficial. For example, if an individual, prior to receiving a pharmaceutical composition comprising an IL-4R antagonist, exhibits a level of an EoE-associated biomarker that signifies the disease state, the individual is therefore identified as a suitable patient for whom administration of a pharmaceutical composition of the invention (a composition comprising an anti-IL-4R antibody) would be beneficial. In related embodiments, the present invention includes methods for treating suitable subjects, wherein a suitable subject may be more susceptible to EoE, for example, due to food allergy, or an atopic disease. For example, the present invention includes methods comprising administering an IL-4R antagonist to subjects who have food allergy, atopic dermatitis, asthma, allergic rhinitis or allergic conjunctivitis. In another example, the present invention includes methods comprising administering an IL-4R antagonist to subjects who have, Mendelian-inherited connective tissue disorders, e.g., Marfan syndrome, Loeys-Dietz syndrome, hypermobile Ehlers Danlos syndrome (EDS) or joint hypermobility syndrome (JHS). Such subject populations may have an elevated level of an EoE-associated biomarker.

According to certain exemplary embodiments, an individual may be identified as a good candidate for anti-IL-4R therapy if the individual exhibits one or more of the following: (i) an eotaxin-3 level greater than about 30 pg/ml, greater than about 40 pg/ml, greater than about 50 pg/ml, greater than about 100 pg/ml, greater than about 1500 pg/ml, greater than about 200 pg/ml, greater than about 250 pg/ml, greater than about 300 pg/ml, greater than about 350 pg/ml, greater than about 400 pg/ml, greater than about 450 pg/ml, or greater than about 500 pg/ml; or (ii) a serum IgE level greater than about 114 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L; or (iii) ≥15 eosinophils per high power field in the esophagus of the subject. Additional criteria, such as other clinical indicators of EoE (e.g., thickening of the esophageal wall, and food allergy indicative of EoE), may be used in combination with any of the foregoing EoE-associated biomarkers to identify an individual as a suitable candidate for anti-IL-4R therapy as described elsewhere herein.

### Interleukin-4 Receptor Inhibitors

The methods of the present invention comprise administering to a subject in need thereof a therapeutic composition comprising an interleukin-4 receptor (IL-4R) inhibitor. As used herein, an “IL-4R inhibitor” (also referred to herein as an “IL-4R antagonist,” an “IL-4Rα antagonist,” an “IL-4R blocker,” an “IL-4Rα blocker,” etc.) is any agent that binds to or interacts with IL-4Rα or an IL-4R ligand, and inhibits or attenuates the normal biological signaling function a type 1 and/or a type 2 IL-4 receptor. Human IL-4Rα has the amino acid sequence of SEQ ID NO: 11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R inhibitors that can be used in the methods of the present invention may function by blocking IL-4-mediated signaling, IL-13-mediated signaling, or both IL-4- and IL-13-mediated signaling. The IL-4R inhibitors of the present invention may thus prevent the interaction of IL-4 and/or IL-13 with a type 1 or type 2 receptor.

Non-limiting examples of categories of IL-4R inhibitors include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R inhibitors also include antigen-binding proteins that specifically bind IL-4 and/or IL-13.

### Anti-IL-4Rα Antibodies and Antigen-Binding Fragments Thereof

According to certain exemplary embodiments of the present invention, the IL-4R inhibitor is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (Vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present invention using routine techniques available in the art. For example, the present invention includes methods comprising the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind IL-4Rα. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4Rα, as used in the context of the present invention, includes antibodies that bind IL-4Rα or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-4Rα may, however, have cross-reactivity to other antigens, such as IL-4Rα molecules from other (non-human) species.

According to certain exemplary embodiments of the present invention, the IL-4R inhibitor is an anti-IL-4Rα antibody, or antigen-binding fragment thereof comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693. In certain exemplary embodiments, the anti-IL-4Rα antibody or antigen-binding fragment thereof that can be used in the context of the methods of the present invention comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-IL-4Rα antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-IL-4R antibody or antigen-binding fragment thereof comprises an HCVR comprising SEQ ID NO: 1 and an LCVR comprising SEQ ID NO: 2. In certain embodiments, the methods of the present invention comprise the use of an anti-IL-4R antibody, wherein the antibody comprises a heavy chain comprising the amino acid sequence of SEQ ID NO: 13. In some embodiments, the anti-IL-4R antibody comprises a light chain comprising the amino acid sequence of SEQ ID NO: 14. An exemplary antibody comprising a heavy chain comprising the amino acid sequence of SEQ ID NO: 13 and a light chain comprising the amino acid sequence of SEQ ID NO: 14 is the fully human anti-IL-4R antibody known as dupilumab. According to certain exemplary embodiments, the methods of the present invention comprise the use of dupilumab, or a bioequivalent thereof. The term “bioequivalent”, as used herein, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In the context of the invention, the term refers to antigen-binding proteins that bind to IL-4R which do not have clinically meaningful differences with dupilumab in their safety, purity and/or potency.

In certain particular embodiments, the methods of the present invention comprise the use of an anti-mouse anti-IL-4R antibody or antigen-binding fragment thereof comprising an HCVR sequence of SEQ ID NO: 9 and an LCVR sequence of SEQ ID NO: 10. In an exemplary embodiment, the methods of the present invention comprise the use of an anti-mouse anti-IL-4R antibody (“anti-mIL-4Rα”) in reducing eosinophilic infiltration of the esophagus in a mouse model of eosinophilic esophagitis.

Other anti-IL-4Rα antibodies that can be used in the context of the methods of the present invention include, e.g., the antibody referred to and known in the art as AMG317 (Corren et al., 2010, Am J Respir Crit Care Med., 181 (8): 788-796), or any of the anti-IL-4Rα antibodies as set forth in U.S. Pat. Nos. 7,186,809, 7,605,237, 7,608,693, or U.S. Pat. No. 8,092,804.

The anti-IL-4Rα antibodies used in the context of the methods of the present invention may have pH-dependent binding characteristics. For example, an anti-IL-4Rα antibody for use in the methods of the present invention may exhibit reduced binding to IL-4Rα at acidic pH as compared to neutral pH. Alternatively, an anti-IL-4Rα antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen binding at acidic pH relative to neutral pH may be obtained. As used herein, the expression “acidic pH” means a pH of 6.0 or less.

### Pharmaceutical Compositions

The present invention includes methods that comprise administering an IL-4R inhibitor to a subject wherein the IL-4R inhibitor is contained within a pharmaceutical composition. The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present invention are disclosed, e.g., in US Patent Application Publication No. 2012/0097565.

### Dosage

The amount of IL-4R inhibitor (e.g., anti-IL-4Rα antibody) administered to a subject according to the methods of the present invention is, generally, a therapeutically effective amount. As used herein, the phrase “therapeutically effective amount” means an amount of IL-4R inhibitor that results in one or more of: (a) a reduction in the severity or duration of a symptom of eosinophilic esophagitis; (b) a reduction in the number of eosinophils in esophagus; (c) prevention or alleviation of an allergic reaction; and (d) a reduction in the use or need for conventional allergy therapy (e.g., reduced or eliminated use of antihistamines, decongestants, nasal or inhaled steroids, anti-IgE treatment, epinephrine, etc.).

In the case of an anti-IL-4Rα antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In certain embodiments, 300 mg of an anti-IL-4R antibody is administered.

The amount of IL-4R inhibitor contained within the individual doses may be expressed in terms of milligrams of antibody per kilogram of patient body weight (i.e., mg/kg). For example, the IL-4R inhibitor may be administered to a patient at a dose of about 0.0001 to about 100 mg/kg of patient body weight.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Anti-IL-4R Antibody Reduces Eosinophilic Esophagitis in an IL-25-Hydrodynamic DNA Delivery (HDD)-Driven Mouse Model

In this Example, the effect of IL-4Rα blockade on eosinophilic esophagitis in an//25-hydrodynamic DNA delivery (HDD) mouse model was assessed. This model is based on the observation that induced IL-25 expression causes IL-13 signaling via the IL-4Rα/IL-13R heterodimer receptor, and consequently results in eosinophilia of the gastrointestinal tract, including eosinophilic infiltration of the esophagus and mucus production.

On Day 0, Balb/c mice were injected with either a plasmid expressing mouse IL25 DNA (“pRG977/m//25,” n=17), or an empty vector (“pRG977,” n=4), each at 25 μg of DNA/mouse by the hydrodynamic DNA delivery (HDD) method (see, e.g., Liu et al. 1999, Gene Therapy 6:1258-1266). The plasmid was diluted in PBS and was injected at a high volume (10% of body weight [ml]), and a high injection rate (6-8 seconds per injection) into the tail vein. Mice that were injected with pRG977/m//25 DNA were treated by subcutaneous (SQ) injections of either an anti-mouse IL-4R antibody (“anti-mIL-4Rα”) or isotype control or a fusion protein of IL-13 receptor alpha unit fused to mouse Fc region (“IL-13Rα2-mFc,” used as a decoy receptor; Yasunaga et al. 2003; Cytokine 24:293-303) on Days 1, 3, 6 and 9 each. Each dose was 50 mg/kg of body weight. The anti-mIL-4Rα antibody used in this Example was an antibody comprising an HCVR with an amino acid sequence of SEQ ID NO:9 and an LCVR with an amino acid sequence comprising SEQ ID NO: 10. The IL-13Rα2-mFc construct had the amino acid sequence of SEQ ID NO: 12. Mice were euthanized on Day 12 for esophageal and blood analysis. Blood was collected, and serum was used to detect total IgE levels by ELISA.

The esophagus harvested from each mouse was fixed, paraffin embedded and stained with hematoxylin/eosin. Sections were scored for pathology and level of eosinophil infiltration was scored as follows:
Score 0: no changes in esophagus wall thickness, no leukocyte infiltrates;
Score 1: low to moderate leukocyte infiltrates detected in sub-mucosa layer;
Score 2: moderate to severe leukocyte infiltrates in sub-mucosa, detectable thickening of the esophagus wall;
Score 3: severe infiltration of leukocytes resulting marked thickening of the esophagus wall.

The proximal, middle and distal part of each esophagus was evaluated by one score, and the final score per animal was an average of these three values.

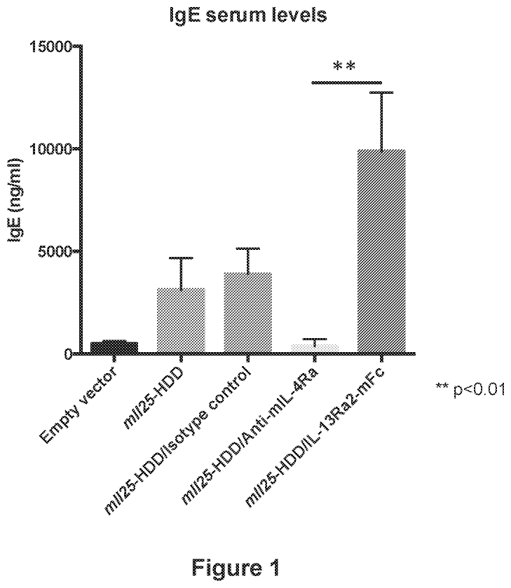

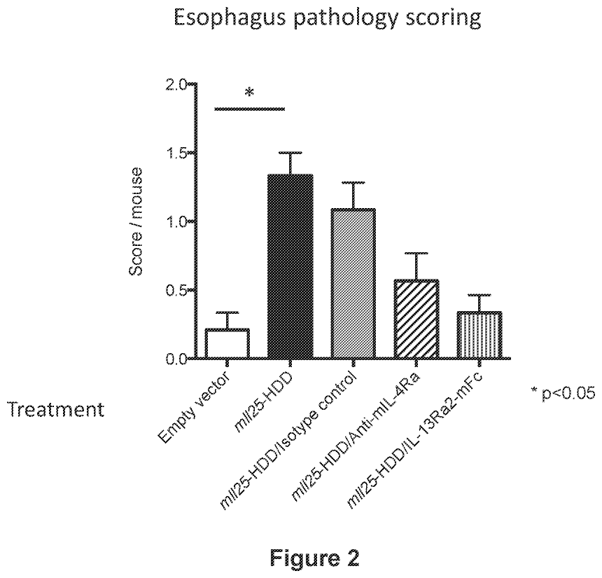

Mice injected with//25 and treated with isotype control antibody or IL-13Rα2-mFc fusion protein showed an increased level of serum IgE which was significantly reduced in the mice treated with anti-mIL-4Rα mAb as compared to IL-13Rα2-mFc treatment (see FIG. 1). Histology scoring results are illustrated in FIG. 2. Both anti-mIL-4Rα mAb and IL-13Rα2-mFc reduced the pathology score of the esophagus by about 50% (see FIG. 2). The t-test was used initially to calculate significance; however, ANOVA (or non-parametric Kruskall-Wallis test) was used for later analysis.

### Example 2: Anti-IL-4R Antibody Reduces Eosinophilic Infiltration of Esophagus in a Mouse Model of Peanut Allergy

In this Example, the effect of IL-4Rα blockade on peanut allergen-induced eosinophilic esophagitis in a mouse model was assessed.

Balb/c mice were sensitized with 200 μg of peanut allergen extract (PAE) in 1 mg of aluminum salts (Alum) adjuvant on day 0 and day 14. Three weeks later, on day 21, mice were challenged intra-nasally with 100 μg of PAE dissolved in 50 μl of phosphate-buffered saline (PBS). The challenge was repeated on day 24, 27 and 30. Starting day 21, one group of challenged mice was not treated and two groups were injected with either anti-IL-4R antibody (“anti-mIL-4R mAb” as described above) at dose 25 mg/kg, or isotype control IgG1. The treatment was applied twice a week, starting day 21. Mice were euthanized 24 hours after the last challenge with PEA on day 31, blood samples and esophagi were collected.

Esophagi were fixed in buffered formalin, paraffin embedded and sectioned slides of tissue were stained with H&E. The extent of leukocyte infiltrates and inflammation was scored in blinded fashion, using following scoring: 0=no changes in esophagus wall thickness, no leukocyte infiltrates; 1=low to moderate leukocyte infiltrates detected in sub-mucosa; 2=moderate to severe leukocyte infiltrates in sub-mucosa, detectable thickening of the esophagus wall; 3=severe infiltration of leukocytes resulting in marked thickening of esophagus wall. Each 25% of the esophagus length received one score (4 scores per esophagus); the average was calculated and used as “score/mouse”.

For differential cell count and activity, the esophagus tissue was digested with Liberase DL enzyme for 30 min at 37° C. (n=5 per group). Following Liberase DL digestion, the cell suspension was filtered and cells were stained with eosinophil-, T-cell-, neutrophil-, and macrophage-specific markers and analyzed by flow cytometry. Some of the cells isolated from esophagi by Liberase DL digest were stimulated with anti-CD3 and anti-CD28 antibodies to activate T-cells, and cultured for 3 days. Tissue culture supernatants were assayed by ELISA for levels of Th2 cytokines (IL-13, IL-10, and IL-4).

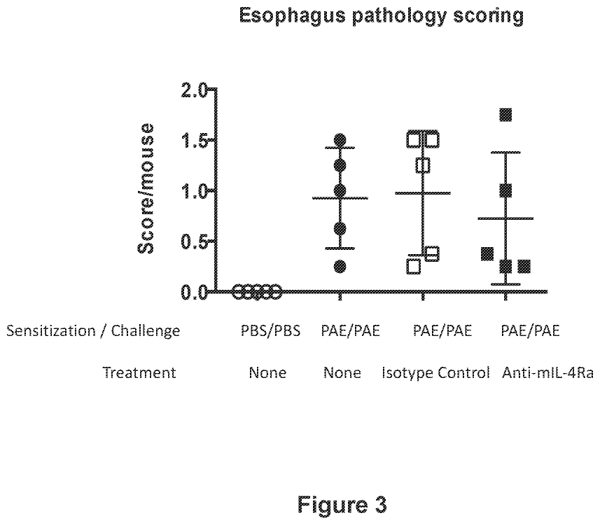

The average score for group of mice that was not sensitized & challenged with peanut allergen was 0. Mice that were sensitized & challenged and not treated scored 0.925±0.497 (mean±SD), mice sensitized & challenged and treated with isotype control or anti-mIL-4R mAb scored 0.975±0.615, and 0.725±0.652, respectively. The differences between isotype control-, anti-mIL-4R mAb—or non-treated groups were not statistically significant, according to one-way ANOVA test (shown in FIG. 3).

Blood was also collected by cardiac puncture post-mortem and the serum analyzed for levels of total IgE and peanut-specific IgG1 (PAE-specific IgG1) levels by ELISA. Briefly, for PAE-specific IgG1 detection, PAE-coated plates were incubated with serially diluted serum samples, following by incubation with anti-mouse IgG1-HRP conjugated antibody. The relative levels of IgG1 serum levels were represented as titer units (OD450 was multiplied by a dilution factor required to achieve OD450≤0.5). For detection of total IgE levels, serially diluted serum samples were incubated with anti-IgE capture antibody on 96-well plates and the IgE was detected by biotinylated anti-mouse IgE secondary antibody. Purified mouse IgE that was HRP-labeled was used as a standard.

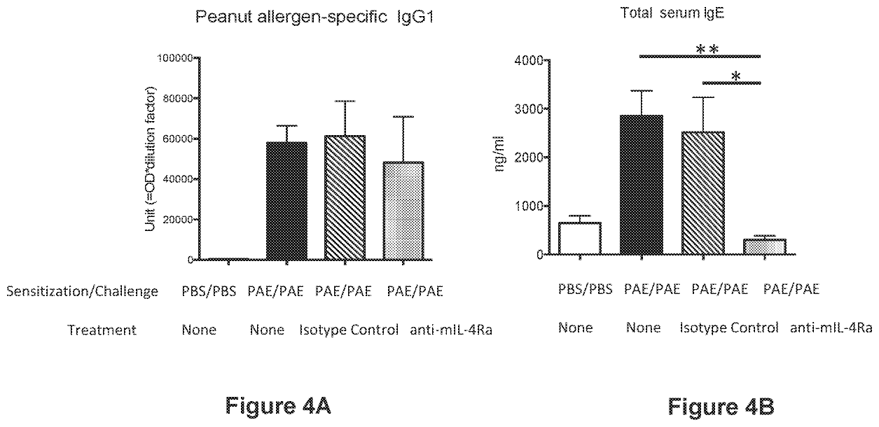

PAE-specific IgG1 and total IgE levels in blood of non-sensitized & non-challenged mice were 439±17.25 U and 644±337.7 ng/ml, respectively. In PAE sensitized and challenged mice with no additional treatment, the PAE-specific IgG1 and total IgE levels increased to 57822±8455 U, and 2857±1149 ng/ml, respectively. Mice treated with isotype control showed 61304±17293 U of PAE-specific IgG1, and 2516±1613 ng/ml of IgE. Treatment with anti-mIL-4Rα mAb did not significantly affect the PAE-specific level of IgG1 (48128±22691 U) but significantly reduced the total serum level of IgE (300±187.8 ng/ml) as compared to either isotype control-treated, or non-treated mice (shown in FIG. 4).

### Example 3: Clinical Trial of Subcutaneously Administered Dupilumab in Adult Patients with Eosinophilic Esophagitis (EoE)

This study is a 32-week, double-blind, randomized, placebo-controlled study to investigate the efficacy, safety, tolerability and immunogenicity of dupilumab in adult patients with EoE. Dupilumab is a fully human anti-IL-4R antibody comprising a heavy chain comprising the amino acid sequence of SEQ ID NO: 13 and a light chain comprising the amino acid sequence of SEQ ID NO: 14; an HCVR/LCVR amino acid sequence pair comprising SEQ ID NOs: 1/2; and heavy and light chain CDR sequences comprising SEQ ID NOs: 3-8.

Study treatment is administered for up to 12 weeks with 16 weeks of safety follow-up. After providing informed consent, eligibility is assessed at the screening visit conducted within 4 weeks of the Day 1 baseline visit. Straumann Dysphagia Instrument (SDI) (0-9) symptom diary data (1 week recall period) and EoE Activity Index (EoEAI) symptom diary data (1 week recall period) are collected during the screening visit and weekly during the study (Straumann et al 2010, Gastroenterology 139:1526-1537). The Adult EoE Quality of Life Questionnaire is collected at the screening visit and end of treatment visit. Patients who meet eligibility criteria will undergo Day 1 baseline assessments. Patients are randomized in a 1:1 ratio to receive subcutaneous dupilumab 300 mg or placebo once weekly during the 12-week double-blind stage.

Patients 18-50 years of age with a history of a diagnosis of EoE confirmed by documented, peak cell density of ≥15 eos/hpf from esophageal histology biopsy specimens at both proximal and distal levels, a history of at least 2 episodes of dysphagia, and a documented history of, or concomitant allergic asthma, allergic rhinitis, atopic dermatitis, or food allergies; or elevated peripheral eosinophil counts (≥300 cells/μL) or elevated serum IgE (≥150 kU/L) are included in the study.

Study drug treatments include a 600 mg loading dose of dupilumab on day 1, followed by a 300 mg weekly dose; or a placebo double dose on day 1, followed by a weekly placebo dose.

The patients will receive 2 injections (including a loading dose) on day 1, followed by weekly injections. At the end of the 12-week double-blind treatment phase, patients are followed for an additional 16 weeks. The study population is stratified by previous response to swallowed topical corticosteroids use. Inadequate response is defined as failure to normalize tissue eosinophils and resolution of symptoms after at least 2 months of topical therapy. Esophageal biopsies are performed at screening and at week 12. Patients who discontinue the study prior to 12 weeks will have the procedure done at their early termination visit. Measurement of inflammatory and remodeling esophageal features based on EoE Endoscopic Reference Score are included as part of the procedure.

All patients receive concomitant medications (except for prohibited medications) as needed, while continuing study treatment. If necessary, rescue medications (such as systemic and topical corticosteroids) or emergency esophageal dilation will be provided to study patients. Patients receiving rescue therapy are discontinued from the study treatment. Safety, laboratory and clinical effect measurements are performed at specified clinic visits. Post-treatment follow-up visits occur at weeks 16, 20, 24 and 28. Samples for DNA and RNA analysis are collected from patients who enrolled in the optional genomics sub-study. Transcriptome sequencing or microarray analysis of the esophageal biopsy RNA will be performed.

The primary objective of the study is to assess the potential efficacy of repeated weekly subcutaneous doses of dupilumab (12 weeks of treatment) compared to placebo, to control EoE in adult patients with active disease. The secondary objectives are: (a) to assess the safety, tolerability and immunogenicity of repeated subcutaneous doses of dupilumab in adult patients with active EoE; (b) to assess the effect of dupilumab on peak eosinophil counts (eos/hpf) in esophageal biopsies; (c) to evaluate the pharmacokinetics of dupilumab in adult EoE patients; (d) to evaluate and optimize clinical endpoint registration endpoint schema under development; and (e) to assess the pharmacodynamic effect of dupilumab using biomarkers including histology and circulating markers (TARC and eotaxin-3).

The patients are monitored for: (a) percent change in Eosinophilic Esophagitis Activity Index (EoEAI) patient reported outcome from baseline to week 12; (b) change in SDI score from baseline to week 12; and (c) reduction of serum TARC and plasma eotaxin-3 from baseline to week 12. The other endpoints monitored include reduction in esophageal hyperplasia, inflammation, inflammatory gene signature and remodeling (histology), and reduction of esophageal mucosa eosinophil counts per high powered field (400×).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1-62. (canceled)

63. A method of treating eosinophilic esophagitis (EoE) in a subject in need thereof, the method comprising subcutaneously administering to the subject an anti-IL-4R antibody,
wherein the anti-IL-4R antibody comprises a heavy chain comprising the amino acid sequence of SEQ ID NO:13 and a light chain comprising the amino acid sequence of SEQ ID NO: 14, and
wherein the anti-IL-4R antibody is administered to the subject at a dose of 300 mg.

64. The method of claim 63, wherein the dose of 300 mg of the anti-IL-4R antibody is administered once a week or once every two weeks.

65. The method of claim 63, wherein the dose of 300 mg of the anti-IL-4R antibody is administered once a week.

66. The method of claim 65, wherein the subject achieves a reduction in the number of eosinophils in the esophagus.

67. The method of claim 66, wherein the subject achieves less than 15 eosinophils per high power field (“eos/hpf”) in the esophagus.

68. The method of claim 67, wherein the subject achieves less than 10, 9, 8, 7, 6, or 5 eos/hpf in the esophagus.

69. The method of claim 68, wherein the subject achieves less than 6 eos/hpf in the esophagus.

70. The method of claim 63, wherein prior to the treatment the subject had ≥15 eos/hpf in the esophagus.

71. The method of claim 70, wherein the dose of 300 mg of the anti-IL-4R antibody is administered once a week or once every two weeks.

72. The method of claim 71, wherein the dose of 300 mg of the anti-IL-4R antibody is administered once a week.

73. The method of claim 72, wherein the subject achieves a reduction in the number of eosinophils in the esophagus.

74. The method of claim 73, wherein the subject achieves less than 15 eos/hpf in the esophagus.

75. The method of claim 74, wherein the subject achieves less than 10, 9, 8, 7, 6, or 5 eos/hpf in the esophagus.

76. The method of claim 75, wherein the subject achieves less than 6 eos/hpf in the esophagus.


SEQUENCE LISTING (14 sequences)

SEQ ID NO: 1 - protein, 124 aa, synthetic construct
  REGION 1..124: note = HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  REGION 1..8: note = HCDR1
GFTFRDYA

SEQ ID NO: 4 - protein, 8 aa, synthetic construct
  REGION 1..8: note = HCDR2
ISGSGGNT

SEQ ID NO: 5 - protein, 18 aa, synthetic construct
  REGION 1..18: note = HCDR3
AKDRLSITIRPRYYGLDV

SEQ ID NO: 6 - protein, 11 aa, synthetic construct
  REGION 1..11: note = LCDR1
QSLLYSIGYNY

SEQ ID NO: 7 - skipped

SEQ ID NO: 8 - protein, 9 aa, synthetic construct
  REGION 1..9: note = LCDR3
MQALQTPYT

SEQ ID NO: 9 - protein, 117 aa, synthetic construct
  REGION 1..117: note = HCVR-mouse surrogate Ab
EVQLQQSGPELVKPGASVRMSCKASGYTFTDYNIHWVKQSHGKSLEWIGYIYPNNGDNGYNQKFRGKATLTVDKSSSTAYMELRSLTSDDSAVYYCARGRLRYFDVWGTGTTVTVSS

SEQ ID NO: 10 - protein, 111 aa, synthetic construct
  REGION 1..111: note = LCVR-mouse surrogate Ab
NIVLTQSPASLAVSLGQRATISCRASESVDNYGHSFMHWYQQKPGQPPKLLIYLASNLESGVPARFSGSGSRTDFTLTLDPVEADDAATYYCQQYNEDPPTFGSGTKLEIK

SEQ ID NO: 11 - protein, 207 aa, Homo sapiens
  REGION 1..207: note = IL-4Ralpha
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 547 aa, synthetic construct
  REGION 1..547: note = IL13Ra2 - mIgG2a, aa 1-311: mouse IL-13Ra2 (E23-S333), aa 312-314: linker, aa315-547: mouse IgG2a Fc (E127 - K359)
EIKVNPPQDFEILDPGLLGYLYLQWKPPVVIEKFKGCTLEYELKYRNVDSDSWKTIITRNLIYKDGFDLNKGIEGKIRTHLSEHCTNGSEVQSPWIEASYGISDEGSLETKIQDMKCIYYNWQYLVCSWKPGKTVYSDTNYTMFFWYEGLDHALQCADYLQHDEKNVGCKLSNLDSSDYKDFFICVNGSSKLEPIRSSYTVFQLQNIVKPLPPEFLHISVENSIDIRMKWSTPGGPIPPRCYTYEIVIREDDISWESATDKNDMKLKRRANESEDLCFFVRCKVNIYCADDGIWSEWSEEECWEGYTGPDSGPGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 451 aa, synthetic construct
  REGION 1..451: note = HC, aa 1-124: HCVR, aa 125-451: HC constant
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 219 aa, synthetic construct
  REGION 1..219: note = LC, aa 1-112: LCVR, aa 112-219: LC constant
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC